SEQUENCE LISTING (7 sequences)

SEQ ID NO: 1 - DNA, 2561 bp, Artificial Sequence
  ARTIFICIAL DNA SEQUENCE
gggagctggttaacctcgagaccggtagcttggactgtaacagacggaagtcgggaagccatgataataatagtaaacaagtttctatttggagatttgtttgaaacgtgataacaaagctataacctttaataattatagactttttgtttctaaatttggacaattctagggtttgttacatgtatctgactttgatcttgcacgggaagcggggtggcagagctccccataattctgaaccagatcgccagaacgagagaagaaaaaaaagaaaaaagtaaaaaaaagagctcctcctatggatctcaaccacgggcaaaaacaaagctcagactgtgtaatccatgctaagtacatgtgaggcaagtactccgtagcgctactcctggcttggctccctacccctccagtcatgatggctgcaaatttcttgggggaggtacacatgtttgttgattggggaggaatgagtaaagtaccctgggcttgaatgaaagcggtaggaggtaggtagagggaggaatttagccttgccttgtcttggccgtaggtttgctgtaatattttttgtctttattatcatcaccacaccaactctacttccttctcctctcctcttcctttctctctctctcttcctcatctctcaacctttcttgaccttcgacgtcaaaacagctttacctccactcacccctctacgcctcgacctttgcgagccccccctccttcgtcgaacgtataatactaccaggaagagcgcacgttcctctgcctcttctgatcatatcatccctttggattattatcgtacggtgcaacactcgtacaaagatatccatatcccctttgtaagtcgacatgctaccgttgcttctgtgctgcacctagatggagacggttggtgtggcttgggccctgagatcccgctcttctgcgcttctctggcaatggagatagggaaatatgtggcaattccggttttggcagatgcatttctggtcttgtttccggacctgcttcagaatgtcgtttgttttcgacccagaaattacgaaccgccggatttgttctcggttcttctggcccgattagcgacaagtctgacgattcttaactcggcccatctattgagctcatgggtcatcctgtgtccttctcactagccaaaccagccgttttgcgtcgcgccgaatgctctgcccaccaaattaccacgccccatccctatcgcctttcacaccacgtcacttaacgtcagatcccacgttagccccccatgccttttctatacctcacgaaagagtctccgttggcctgcataacaagtgtccgcagaacagcctgatatccatccctgttatcagtagtgggatctacaatcgttcgatcaattgagatatatatgtgtgctggttgtccaatctgcaccacacctgttgtctcatcatcttcattcctttcgcagccagagctaccccgccaagctcttctctcacaaacaacttgctaaccacattcacaatgatttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgt

SEQ ID NO: 2 - DNA, 2428 bp, Artificial Sequence
  ARTIFICIAL DNA SEQUENCE
gggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagcagtttcatg

SEQ ID NO: 3 - DNA, 9717 bp, Artificial Sequence
  ARTIFICIAL DNA SEQUENCE
cgacagagtcctaggagttgcgttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacctcgagaccggtagcttggactgtaacagacggaagtcgggaagccatgataataatagtaaacaagtttctatttggagatttgtttgaaacgtgataacaaagctataacctttaataattatagactttttgtttctaaatttggacaattctagggtttgttacatgtatctgactttgatcttgcacgggaagcggggtggcagagctccccataattctgaaccagatcgccagaacgagagaagaaaaaaaagaaaaaagtaaaaaaaagagctcctcctatggatctcaaccacgggcaaaaacaaagctcagactgtgtaatccatgctaagtacatgtgaggcaagtactccgtagcgctactcctggcttggctccctacccctccagtcatgatggctgcaaatttcttgggggaggtacacatgtttgttgattggggaggaatgagtaaagtaccctgggcttgaatgaaagcggtaggaggtaggtagagggaggaatttagccttgccttgtcttggccgtaggtttgctgtaatattttttgtctttattatcatcaccacaccaactctacttccttctcctctcctcttcctttctctctctctcttcctcatctctcaacctttcttgaccttcgacgtcaaaacagctttacctccactcacccctctacgcctcgacctttgcgagccccccctccttcgtcgaacgtataatactaccaggaagagcgcacgttcctctgcctcttctgatcatatcatccctttggattattatcgtacggtgcaacactcgtacaaagatatccatatcccctttgtaagtcgacatgctaccgttgcttctgtgctgcacctagatggagacggttggtgtggcttgggccctgagatcccgctcttctgcgcttctctggcaatggagatagggaaatatgtggcaattccggttttggcagatgcatttctggtcttgtttccggacctgcttcagaatgtcgtttgttttcgacccagaaattacgaaccgccggatttgttctcggttcttctggcccgattagcgacaagtctgacgattcttaactcggcccatctattgagctcatgggtcatcctgtgtccttctcactagccaaaccagccgttttgcgtcgcgccgaatgctctgcccaccaaattaccacgccccatccctatcgcctttcacaccacgtcacttaacgtcagatcccacgttagccccccatgccttttctatacctcacgaaagagtctccgttggcctgcataacaagtgtccgcagaacagcctgatatccatccctgttatcagtagtgggatctacaatcgttcgatcaattgagatatatatgtgtgctggttgtccaatctgcaccacacctgttgtctcatcatcttcattcctttcgcagccagagctaccccgccaagctcttctctcacaaacaacttgctaaccacattcacaatgatttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgat

SEQ ID NO: 4 - DNA, 9622 bp, Artificial Sequence
  ARTIFICIAL DNA SEQUENCE
gggccgcacaggtctgtagatgcataatcttagagacaagttgacttaattaacgcaactcagctccctccaacctccgagcgggtacaagattcccgcatcgcactacagttcgtagcgccagtctaccagtctgccagctgcagctgcagatgcagttgcagggttgggggtagccctgtcgcaccgacgggtgtacagacttggttacgcaaaagcctgcactcaagtgggaaatggttacaggctcaggttacgtacagacattcagtttcaactgcctattccgaagtaattatccttgttcaactgttcattctaacaaaacacgaccgaactatttgtaatcattataacagaccaccattctccaaacgagtatccgtacccgcaaaacatacttccaaactcgccttcggttagttgtagcgacgcgtggtcttgctccactgctcattgccgctcatctggtactctaggcccttcatatacgccttggcagcttcagagcagccgtctggggatgcgatcttctctgccacgatctcgtcatgcacagtctcccactcccgagtcatagaccaaagcacgcgtttcgttgcgggtataccaagcttggactcctccgccagaaccttcacagctgaacaaaggaaggctccctccttgtgtccagtccgagacgcttcctcttccttgtcataactgtatatgtcattgactacagaaagctgcttggcacagttggcttcaagggctttcatatcctgaagctcatctgcactgagtctaagtcccatcgagaatctcattagagccgaaagcaacgccttgcctacatccttctcacggtactcgagataatgccccaattcatggatgctcaaacgcgctcgatctgtctgcgcgcgcatgaacacgaaagtcggctcaagaacttcgttggccagctccgcatcatgggcgcgcatgctttcccagaggtcatagagaatgaactcttctggcttggttcggtccgggagcacgtctccacgcgatatcgggatcaatctgttgttgtaggcttctccatccgcgaaagacatatgctcaagaacatcatcgataaggaagagaacggtcagcagtcggcaggcaaagtggatgcgatcgtccagtgcaagagggaagtaaagacaagtaacctctgagaacttggcatccaagaaggtgcgaacagccttgaagctgggaaatttccagttctccaggaagtatccatcgacttcgtcctgtacctccttcactcgtggatggcaaaggtacgaccactgagtaggaggcggagtgatcgtcgtgtcatttctgggcctgattgtctctttgaccagagggctattgatagggttttcaaggtgcacgaacggttgggccagggaagaaatggtttctgttgaggtagccatattggccaaggtgtattggtaacaattcttgatgaatcaacaaactagatcgctatcattgtcttataacaaagtgccgttgctaccaactatatatcattagatggcaaccatctcgaaccgtacactattgctagttgacattttcttgttaggcctaaagactgagccaatatcgatgccaacagcctccccaaccggctacctgtgacatagaccgtgacgtaggcttgatccccctcgtctcatgcaaaactacgaagttgagaaagacttcggctcccagaaggaaggattagtccggtttgtctttagctcgaagagcatttggtttagctacgcaagcgattggtcctggatcctggtgtcaatggacgaggagtaggcctgaagctttgagttcggttgttgtccgtaactgagtttgaactagtggaatagccttctgtcgataggggaggaagcctcacttgtccaagcagccacggagttattttgtgccctgtcaaaagcttaataaggcgaaaggccttaagtgtaaccacttttatgatataccctgcaaacccgtcggctgttacacatattcaaaaccgttcgttcccggatgtactgcaaccgcatctttgaataaggttcaccaacttagccttgtttagttagcgggaggctctgaatttagctgtactatccttgaacccttcctttgtgcgggaatctgtactaagatctccatcacctgtgtctaaaccgcgtatctagcattgtgttctatacacgaactagaacacagatgtcctcggctgtgccgtagataaatcttgttttcgtatttgacatagataacttaacttgcgctaaatggtgattattctccgttgtcgacgcagtacggaattccgaatcccatccttagtcgtactcgtcatagtactgggagattgtccatctaggcaggtgttggagattgagaggtttcttccttcttgacacgtgcttactagtcgcatcggatcccgggcccgggttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacggg

SEQ ID NO: 5 - DNA, 31 bp, Artificial Sequence
  ARTIFICIAL DNA PRIMER
cacactgcagcgataccgagatagcaactac

SEQ ID NO: 6 - DNA, 44 bp, Artificial Sequence
  ARTIFICIAL DNA PRIMER
cacactgcagcaattgaacgttggcagcctgctcatagaacttg

SEQ ID NO: 7 - DNA, 9967 bp, Artificial Sequence
  ARTIFICIAL DNA SEQUENCE
tcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattccgatacggggaatcgaaccccgagctgctgtgcacatgcaatgagagacagcgatgttaaccattacaccatatcggatgttatatttttattctcctcaaataaatgtatataactataggaatggattcaatagaatctcccaggtcagtgtacttactccggaccctcttaaatatgtcgccagagtgcttcacttgaaccgtaattagagcaggtatcgcgcaaattttacaggcatttgagacacttttttgtcctggtgagagtttatatatcgcataacaagcccttgtgctggacatctgctaatgtgagggaagcacgagaccgccgtcagactgaacgctacgtttgaccataatctaaatatacgctacctatatttgtcgctatggctactatttccgatgttaccgaagaagagattttataaaacctggtgaagtcgaggaatcgatgagctcgcgaaataggtggggtatcttgcacatacttctacttgggtatatccccatggctattctcactactaagtctctggtgagaggggattgaagcaggaactgccagagctacgtcggtgcttccagtcttgtttgcatcatgaactccaccgcgtccagttgccactactcaggaatgtacttcagagactttttcgagaattttgtggatttacgctttccttgggccgacctgactcccacctgactatcttggattatcttgcaaagccagctgccaaccgaggtttccactgtaaaatatgaccctaactcagcacacgtgacctccaggttgtgcttcagtagctgtacttaatcgtctgtatttcgttaactatgcttagcacatacatatatatcatggcactcggttgattagtctccgagaggttctacttttctttattgatgccgtggcaccgcaggacagtctgcacgttactgcttctgtgtgtatctacagcgacaaagtagtcctgataacaggagcttcagatgccaaggtggtaggagcgctccactaggatggaaaatgtcctcctaggacaacaatataaaagcggtactcttactgatgtctattggaagaaaacttggggactcgacacagtaataaggacaatagacagacgcggaaatcacctgcaaggctgagctatcgttattactctaccgcaaggcaacaaccagctcacccctgaggcacgggtaccatgggttgagtggtatggggccatccagagtcacctgtggcagcatgagactgcactcgaagcagccatcaacccagccaatattctgggctttccatccttagatcacatttgagatataacccatttggtgagagacacttgtgccgttatacgtgtctagactggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagatctagaaaccgcaatctctatgaaatgataaggtgcttgaccaattctaatatcggtgtggtggactgtcctaacaaaacccggctatattggacccatcagagctctctttgatcttcatatccagcgaatcgagtcgcagcatttacatcaggcttgattgagtatacatcagcgtttgcagttggactggatcaggaggcaacacctcatgttgtagttcatcatctaggtcttttgaaacggactactgggcttgtcataccaagtgcatcttagcgcttgtttgtgtgacctattgtatcatgggtaacatgttgatgtcattcaggctgagtaatctgcctatccaatgatgactagttaaagatggatatacgtcgtacatccgaccttcagggccggatgcagcgataccgagatagcaactacactcgagaagagcgtatcgagaatttgcactatgcttataacaaggctgctcatcactttgctcagccacgccaacagcagctactcaaggtagaccccaagcgactacaggcttccctccaaacaatcgttggcatggtcgtgtacagttgggcaaaggtctctaaagaatgcatggccgatctatctatacattatacctacactctcgttttggacgatagcaaggatgacccgtacccaaccatggtaaactatttcgacgatcttcaagctggacgcgaacaagcccatccgtggtgggcactcgtcaacgagcactttcccaatgtccttcggcactttggtcccttctgctcattgaaccttattcgcagcactctggactgtaagtaccccggccttgttattcctccacatcccgaagttgacaatgatggaattatagttttcgagggatgctggatcgaacagtacaactttggaggatttccaggatctcatgattaccctcagtttcttcgacgtatgaacggcttgggtcattgtgtcggggcttctctatggcccaaggagcagttcaatgagcgaagtctattccttgagattacatcagccattgcccagatggagaactggatggtctgggtcaatgatcttatgtcattctacaaggaattcgacgatgagcgtgaccagatcagtctcgtcaagaactatgtcgtctctgatgagatcagcctccacgaagccttggagaaactcacccaagacactctgcactcgtccaaacagatggtagctgtcttctcggacaaagaccctcaggtgatggacacgattgagtgcttcatgcacggctatgtcacgtggcacttgtgcgatcgcaggtaccgccttagcgagatatacgaaaaggtcaaagaagagaagactgaggacgctcagaagttctgcaagttctatgagcaggctgccaacgttcaattgctgcattcttcagcatacgaaactggtgttataactttgaatcccatcaatggttatcgggttggtcccataataaacaaaaagaggctgcattgccctatggcatgccattcttgacgcggcatgccgagttagccgaaattgggccaatcggcttcccgtcagaagaggatcactggcgcctcaattttggtttttatagtaccggtgcagcacggctaaacgttctctttctacggcccggcatctcataagcagccaggtacagaaaagcaagaatagaaaattcaggaaaaaccatcccagcatccaacctggtgcccagaatggcggaaacgtcggatataaggagcaatggctatccattaccgactgaacttttgcaggagatactcctattcgctgacttccagtcgttcttttcggcgagttggacgtgcaaagattggcggaacgcggcgttaagctcttatgtacttcggcaccagctcaacactgttcccactgttcccacactcgccgaggcagacattgaacaggcgacaccacgagaactgagaatactattccaccgtgtctgccggcagaacctcatggggatacggagcaacgtttccctcagcaatacggaggagaaaacagtaagaccaatgtcggctattgcggttcaatctcggcatgggtgccaatacgcgcagttgcgtgggatgacattcatatcaagacg

(19) United States
(12) Patent Application Publication
(10) Pub. No.: US 2013/0224817 A1
(43) Pub. Date: Aug. 29, 2013

(54) Filamentous Fungi and Methods for Producing Isoprenoids

(71) Applicant: Thomas Hohn, Chapel Hill, NC (US)

(72) Inventor: Thomas Hohn, Chapel Hill, NC (US)

(73) Assignee: NOVOZYMES A/S, Bagsvaerd (DK)

(21) Appl. No.: 13/819,198

(22) PCT Filed: Nov. 1, 2011
(86) PCT No.: PCT/US11/58700

§ 371 Date: Feb. 26, 2013

Related U.S. Application Data
(60) Provisional application No. 61/408,679, filed on Nov. 1, 2010.

(51) Int. Cl. C12P 5/00; C12N 15/80

(52) U.S. Cl. CPC C12P 5/007; C12N 15/80
USPC 435/166; 435/254.7

(57) ABSTRACT

The present invention relates to the production of a isoprenoid products from a lignocellulosic feedstock. Specifically at least triple mutant of filamentous fungi having the isoprenoid pathway results in production of isoprenoid products in commercial quantities. One embodiment of the invention relates to producing the isoprenoid products at the site of the lignocellulosic feedstock to reduce costs of shipping the feedstock.

The application claims priority of U.S. provisional application No. 61/408,679 filed on Nov. 1, 2010 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the production of isoprenoid products from a filamentous fungus using a biomass feedstock such as a lignocellulosic feedstock. In particular, the present invention relates to a filamentous fungi having the trichothecenes pathway and method for producing isoprenoid products using biomass feedstock wherein the fungus is a mutant fungus having no or low Tri5 expression or Tri5 suppression and increased expression of a terpene synthase and at least one of Tri6 or Tri10.

2. Description of Related Art

Isoprenoids are widely distributed in nature and represent a diverse family comprising over 30,000 compounds. Some isoprenoids perform essential cellular functions involving cell metabolism and membrane integrity while others perform important functions in the ecology of plants and microoganisms. Numerous commercial products contain isoprenoids including pharmaceuticals, cosmetics, perfumes, pigments and colorants, fungicides, antiseptics, nutraceuticals, biofuels, and fine chemical intermediates. Because of their importance in biological systems and broad use in commercial applications, isoprenoids have been intensely studied by scientists.

Current methods for obtaining isoprenoids include chemical extraction from biological materials (e.g., plants, microbes, and animals) and partial or total organic synthesis in the laboratory. Both approaches are often found to be unsatisfactory. Extraction of isoprenoids from biological materials may require the use of toxic solvents and result in low yields due to low concentrations of the isoprenoid product in the source material. Organic synthesis of isoprenoids is typically inefficient and complex requiring several steps to obtain the desired product. These steps often involve the use of toxic solvents, which require special handling and disposal procedures. Difficulties in obtaining the required amounts of isoprenoids for commercial and scientific applications have limited their practical use. For example, the inability to obtain sufficient quantities of certain isoprenoids has slowed down the progression of drug candidates through clinical trials, and the use of certain promising isoprenoid drug candidates as pharmaceuticals have also been deterred due to concerns that the costs related to synthesis of the drug may not support its commercial production. Another promising application for isoprenoids is their use as biofuels but this application also requires low cost and large scale production of C₁₀ (ten carbon atoms) and C₁₅ (fifteen carbon atoms) isoprenoid hydrocarbons beyond what is currently possible.

In order to solve isoprenoid product supply problems, researchers have looked to the biosynthetic production of isoprenoids in microbes. Isoprenoid products are typically composed of repeating five carbon isopentenyl diphosphate (IPP) units and are synthesized by consecutive condensations of the precursor IPP units and its isomer dimethylallyl pyrophosphate (DMAPP). In Fungi, the mevalonate-dependent (MEV) pathway converts acetyl coenzyme A (acetyl-CoA) to IPP, which is subsequently isomerized to DMAPP. Condensations of IPP and DMAP units lead to the synthesis of geranyl pyrophosphate (GPP, C₁₀), farnesyl pyrophosphate (FPP, C₁₅), and geranylgeranyl pyrophosphate (GGPP, C₂₀) which serve as intermediates for numerous isoprenoid products. The elucidation of the MEV pathway has enabled the biosynthetic production of isoprenoids using microbial host systems. For instance, portions of or the entire MEV pathway have been engineered into microbes, such as Escherichia coli and yeast resulting in the production of a foreign isoprenoid product called amorpha-4,11-diene.

Despite recent progress in the engineering of isoprenoid production in microbes, the large quantities of isoprenoid products needed for many commercial applications require expression systems and fermentation methods that produce even more isoprenoids than can be accomplished with the current technologies. A key feature of the successful redirection of microbial metabolism toward isoprenoid production requires that the isoprenoid biosynthetic pathway be appropriately engineered to permit up-regulation of the isoprenoid pathway genes. The present invention addresses this need and provides related advantages as well. Specifically, the current invention is directed toward identification of new methods for the synthesis of isoprenoid products in certain fungal species with native terpene biosynthetic pathways.

### BRIEF SUMMARY OF THE INVENTION

The present invention relates to the discovery that a filamentous fungus having the trichothecene biosynthesis pathway which has a lower, non-functioning or inhibited (chemically or biologically) Tri5 gene alone with an augmented terpene synthase gene and one or more augmented gene products from the group of Tri6 and Tri10 produces an improvement in the production of an isoprenoid product.

Accordingly, in one embodiment of the present invention there is a mutant isoprenoid producing filamentous fungus having the trichothecene pathway comprising:
a) a disrupted Tri5 gene or a mutant Tri5 gene having low trichodiene synthase production;
b) a modified nucleic acid sequence encoding for a terpene synthase gene having isoprenoid production; and
c) a modified nucleic acid sequence encoding for at least one of the genes selected from the group consisting of Tri6 and Tri10 the sequence modified such that the filamentous fungus produces at least 10% more isoprenoid product than the parent filamentous fungal cell when cultured under the same conditions.

In another embodiment of the present invention there is a mutant isoprenoid producing filamentous fungus having the isoprenoid pathway comprising:
a. a modified nucleic acid sequence encoding for at least one of the genes selected from the group consisting of terpene synthase, Tri6 and Tri10, the sequence modified to increase the production of the gene product; and
b. the presence of a Tri5 inhibitor sufficient to inhibit at least a portion of the Tri5 gene product;
wherein the filamentous fungus produces at least 10 percent more isoprenoid than the parent filamentous fungal cell when cultured under the same conditions.

In another embodiment of the present invention there is a method of producing isoprenoids comprising:
a. Selecting a mutant filamentous fungus having the trichothecenes pathway comprising:
   i. one or more of a disrupted Tri5 gene, a mutant Tri5 gene having low trichodiene synthase production and the fungus in combination with a Tri5 gene product inhibitor;
   ii. a modified nucleic acid sequence encoding for at least one of the genes selected from the group consisting of terpene synthase, Tri6 and Tri10, the sequence modified to increase the production of the gene product,
b. cultivating the mutant filamentous fungus using a growth media selected from the group comprising a sugar, a starch, a cellulose and a hemicelluloses; and
c. isolating isoprenoids from growth media;
wherein the filamentous fungus produces at least 10 percent more isoprenoid than the parent filamentous fungus when cultured under the same conditions and using the same growth media.

### BRIEF DESCRIPTION OF THE DRAWINGS

FIG. 1 is a diagram of the trichothecene pathway showing the relationship of Tri5, Tri6 and Tri10.

FIG. 2 is a diagram of the biosynthetic pathway showing isoprenoid biosynthesis in filamentous fungi.

FIG. 3 shows a map of expression plasmid pDOR103.

FIG. 4 shows a map of expression plasmid pDOR311.

FIG. 5 shows a map of expression plasmid pDOR320.

FIG. 6 shows a map of expression plasmid pDOR318.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The term “about” means±10 percent.

The term “substantially” means±10 percent.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

The term “operably linked” refers to a juxtaposition of biological components on a single DNA molecule that are in a relationship permitting them to function in their intended linked manner. For instance, a promoter is operably linked to a nucleotide sequence if the promoter affects the transcription or expression of the nucleotide sequence.

The term “mutant” refers to cells related to a parent cell by a modification of one or more genes involved in the production of trichothecenes, e.g. disruption or deletion of the Tri4 gene such that the Tri4 gene no longer functions. Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues. When such agents are used, the mutagenesis is typically performed by incubating the parent cell to be mutagenized in the presence of the mutagenizing agent of choice under suitable conditions, and selecting for mutant cells exhibiting reduced or no expression of the gene.

Modification or inactivation of the gene may be also accomplished by introduction, substitution, or removal of one or more nucleotides in the gene or a regulatory element required for the transcription or translation thereof. For example, nucleotides may be inserted or removed so as to result in the introduction of a stop codon, the removal of the start codon, or a change of the open reading frame. Such a modification or inactivation may be accomplished by site-directed mutagenesis or PCR generated mutagenesis in accordance with methods known in the art. Although, in principle, the modification may be performed in vivo, i.e., directly on the cell expressing the gene to be modified, in one embodiment the modification be performed in vitro as exemplified below.

Alternatively, modification or inactivation of the gene may be performed by established anti-sense techniques using a nucleotide sequence complementary to the nucleic acid sequence of the gene. More specifically, expression of the gene by a filamentous fungal cell may be reduced or eliminated by introducing a nucleotide sequence complementary to the nucleic acid sequence of the gene which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated.

The term “Filamentous fungi” includes all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In the methods of the present invention, the filamentous fungal cell may be a wild-type cell or a mutant thereof. Furthermore, the filamentous fungal cell may be a cell which does not produce any detectable trichothecene(s), but contains the genes encoding a trichothecene(s). Preferably, the filamentous fungal cell is an Acremonium, Aspergillus, Aureobasidium, Cryptococcus, Filibasidium, Fusarium (e.g. F. gramineareum, F. sporotrichioides, F. venenatam) Gibberella, Humicola, Magnaporthe, Mucor, Myceliophthora, Myrothecium, Neocallimastix, Neurospora, Paecilomyces, Penicillium, Piromyces, Stachybotrys, Schizophyllum, Talaromyces, Thermoascus, Thielavia, Tolypocladium, Trichoderma, or Trichothecium cell.

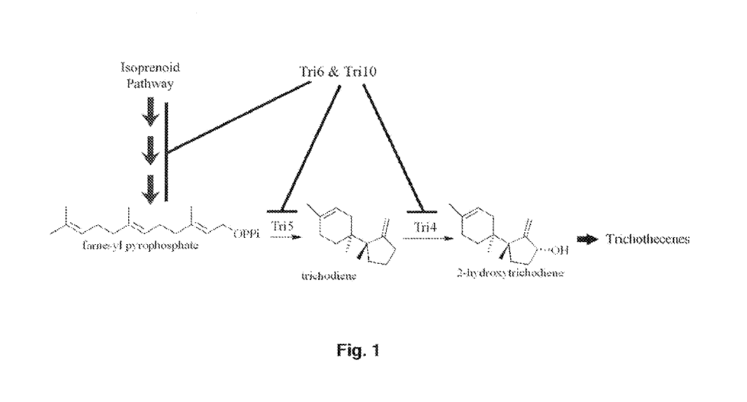

The term “trichothecenes” is defined herein as a family of sesquiterpene epoxides produced by a sequence of oxygenations, isomerizations, cyclizations, and esterifications leading from trichodiene to the more complex trichothecenes. The trichothecenes include, but are not limited to, 2-hydroxytrichodiene, 12,13-epoxy-9,10-trichoene-2-ol, isotrichodiol, isotrichotriol, trichotriol, isotrichodermol, isotrichodermin, 15-decalonectrin, 3,15-didecalonectrin, deoxynivalenol, 3-acetyldeoxynivalenol, calonectrin, 3,15-diacetoxyscirpenol, 3,4,15-triacetoxyscirpenol, 4,15-diacetoxyscirpenol, 3-acetylneosolaniol, acetyl T-2 toxin, and T-2 toxin; and derivatives thereof. The trichothecene biosynthetic pathway is shown in FIG. 1 (Microbiol. Rev., 57: 595-604).

The “isoprenoid” refers to organic compounds having two or more units of hydrocarbons, wherein each unit consists of five carbon atoms. Usually there are isoprene derivatives of branched chain unsaturated hydrocarbons.

The term “constitutively active” refers to a promoter that is expressed and not known to be subject to regulation completely ceasing expression; that is, it is always “on,” and does not entirely rely on activation by some other biological system.

The term “inducible” or “inducibly active” refers to a promoter whose activity level increases in response to treatment with an external signal or agent.

The term “nonrevertable site-selected deletion” refers to the deletion a significant amount of the Tri4 DNA sequences such that the organism is incapable of reversion to the wild type. Reversion is a finite probability over time that exists with naturally occurring or induced point mutations wherein the single mutations could easily and naturally mutate back during production use to produce active gene product. Deletions of the invention include large deletions or active site deletions involving a single codon for an active site residue.

The term “gene product” refers to RNA encoded by DNA (or vice versa) or protein that is encoded by an RNA or DNA, where a gene will typically comprise one or more nucleotide sequences that encode a protein, and may also include introns and other non-coding nucleotide sequences.

The term “at least 10 percent more isoprenoid” refers to an increase in the quantity of isoprenoid produced by a fungal cell as measured by chemical analytical methods and expressed as grams isoprenoid per liter of culture or grams isoprenoid per gram fungal culture dry weight when comparing the modified strain to a parent or wild type strain. In some embodiments, the increase in the quantity of isoprenoid is at least 15 percent more isoprenoid, e.g., at least 20 percent more, at least 30 percent more, at least 40 percent more, or at least 50 percent more.

The terms “enzymatic activity” or “catalytic activity” refer to the ability of the Tri5 gene product to catalyze the required chemical transformation of trichodiene so as to produce a trichodiene product.

The term “low trichodiene synthase” refers to the amount of enzymatically active Tri5 gene product produced in a Tri5 mutant strain or Tri5 inhibited strain such that the levels of trichodiene produced are more than 10 percent less than are observed in the parent or wild type strain by chemical analysis under the same growth conditions.

The term “autonomous maintenance” refers to a DNA or vector that replicates within a filamentous fungal cell independently of the chromosomal DNA. For autonomous replication, the DNA or vector may further comprise an origin of replication enabling the vector to replicate autonomously in the filamentous fungal cell in question.

The term “promoter” refers to a portion of a gene containing DNA sequences that provide for binding of RNA polymerase and initiation of transcription and thus refers to a DNA sequence capable of controlling expression of a coding sequence or functional RNA. Promoter sequences are commonly, but not always, found in the 5′ non-coding regions of genes, upstream of one or more open reading frames encoding polypeptides. Sequence elements within promoters that function in the initiation of transcription are often characterized by consensus nucleotide sequences. A promoter sequence may include both proximal and more distal upstream elements. A promoter may be, for example, constitutive, inducible, or environmentally responsive.

The term “terminator” refers to a sequence recognized by a filamentous fungal cell to terminate transcription. The Tri5 terminator sequence is operably linked to the 3′ terminus of the nucleic acid sequences encoding the Tri6 or Tri10 polypeptides. Any terminator which is functional in the filamentous fungal cell may be used in the present invention.

The term “inhibitor” refers to, for purposes of this invention, a substance that prevents an enzymic process as a result of the interaction of the substance with the enzyme so as to decrease the rate of reaction.

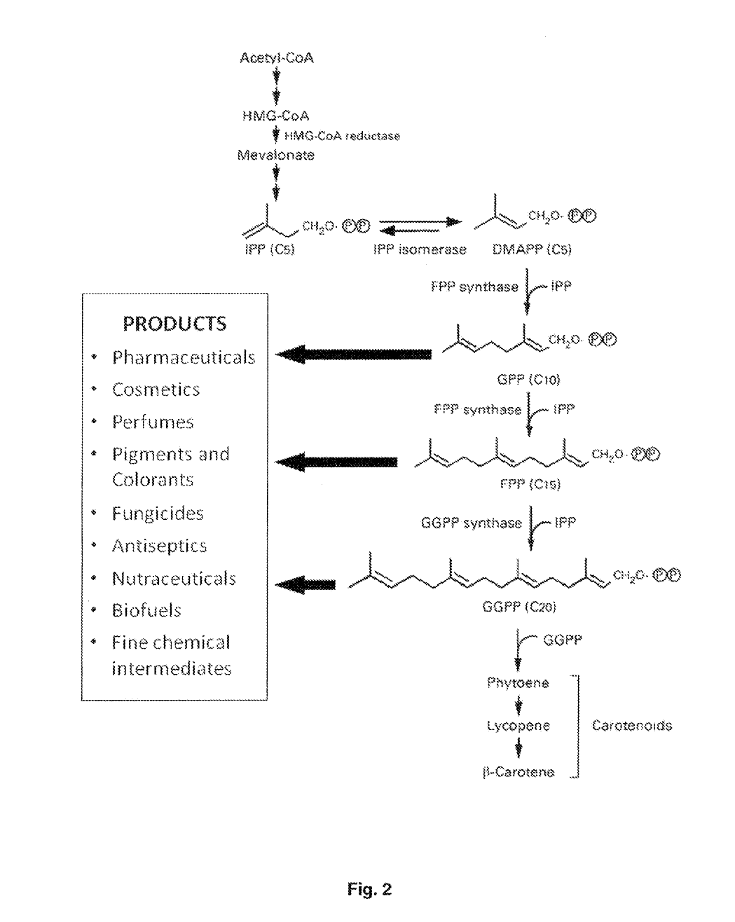

The term “trichothecene pathway” is used herein to refer to the biosynthetic pathway that converts farnesyl pyrophosphate (FPP) to trichothecenes. The first two steps in the trichothecene pathway are illustrated schematically in FIG. 2.

The term “glucose equivalent” is used to describe the degree of hydrolysis of starch or cellulose into glucose monomers or the percentage of the total solids that have been or can potentially be converted to reducing sugars.

The term “biomass” refers to any biological material that can be used for biofuel or bioproduct industrial processes including, but not limited to, lignocellulose, algae, algal process wastes, chitin, chitosan, pectins (including sugar beet process residues), and proteins (including oil seed crushing residues). Other materials are known in the art and can be identified by one skilled in the art.

The term “lignocellulosic feedstock” refers to use of plant biomass composed of lignocellulose (cellulose, hemicellulose, and lignin) as a feedstock for biofuel and bioproduct industrial processes. The carbohydrate polymers of lignocellulose (cellulose and hemicelluloses) are tightly bound to the lignin and are not readily accessible to enzymatic hydroloysis. Lignocellulosic feedstocks include, but are not limited to, agricultural residues (including corn stover, wheat straw, and sugarcane bagasse), energy crops (including sorghum, switchgrass and miscanthus), wood residues (including sawmill and paper mill discards), forestry wastes, industrial wastes (including paper sludge), and municipal paper and landscape waste. Other materials are known and can be identified by one skilled in the art.

The term “vector” refers to a nucleic acid sequence or molecule (e.g. a plasmid) that transduces, transforms, or infects a host strain, thereby causing the cell to produce nucleic acids and/or proteins other than those that are native to the cell, or to express nucleic acids and/or proteins in a manner that is not native to the cell. Alternatively, the vector may contain additional nucleic acid sequences for directing integration by homologous recombination into the genome of the filamentous fungal cell. The additional nucleic acid sequences enable the vector to be integrated into the genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequences that are homologous with the target sequence in the genome of the filamentous fungal cell. Furthermore, the integrational elements may be non-encoding or encoding nucleic acid sequences. On the other hand, the vector may be integrated into the genome of the cell by non-homologous recombination.

The term “growth media culture” refers to cultivation in a nutrient medium suitable for production of trichodiene using methods known in the art. For example, the cell may be cultivated by shake flask cultivation, or small-scale or large-scale fermentation (including continuous, batch, fed-batch, or solid state fermentations) in laboratory or industrial fermentors with a suitable medium and under conditions allowing the trichodiene to be secreted and/or isolated. Suitable nutrient media comprising carbon and nitrogen sources and inorganic salts are available from commercial suppliers or may be prepared using biomass as the medium carbon source. Those skilled in the art can produce appropriate cultures with minimal experiments in view of the present invention.

The term “parent strain” refers to a strain of microorganism that is mutated, electroporated, or otherwise changed to provide a strain or host strain of the invention, or a strain that precedes a strain that has been mutated, electroporated, or otherwise changed to provide a strain or host strain of the invention. In one embodiment it refers to a naturally occurring strain.

The term “modified nucleic acid sequence” refers to a nucleic acid molecule, either single- or double-stranded, which is isolated from a naturally occurring gene or which has been modified to contain segments of nucleic acid which are deleted, combined, and/or juxtaposed in a manner which would not otherwise exist in nature.

The word “pyrophosphate” is used interchangeably herein with “diphosphate”.

The term “host strain” is used herein to refer to any archae, bacterial, or eukaryotic living cell into which a heterologous nucleic acid can be or has been inserted. The term also relates to the progeny of the original cell, which may not necessarily be completely identical in morphology or in genomic or total DNA complement to the original parent, due to natural, accidental, or deliberate mutation.

The term “transformation” refers to a permanent or transient genetic change induced in a cell following introduction of a new nucleic acid. Genetic change (“modification”) can be accomplished either by incorporation of the new DNA into the genome of the host strain, or by transient or stable maintenance of the new DNA as an episomal element. In eukaryotic cells, a permanent genetic change is generally achieved by introduction of the DNA into the genome of the cell.

The Trichothecene biosynthetic pathway in filamentous fungi is fairly well known. The depiction in FIG. 1 outlines the trichodiene synthetic pathway as well as its place in the isoprenoid biosynthetic pathway. The FIG. 2 chart depicts the known isoprenoid product production using the pathway that is the focus of the present invention. The isoprenoid pathway is present in all fungi while the trichothecene pathway exists in a number of filamentous fungi including, but not limited to, species such as Acremonium, Aspergillus, Aureobasidium, Cryptococcus, Filibasidium, Fusarium, Gibberella, Humicola, Magnaporthe, Mucor, Myceliophthora, Myrothecium, Neocallimastix, Neurospora, Paecilomyces, Penicillium, Piromyces, Stachybotrys, Schizophyllum, Talaromyces, Thermoascus, Thielavia, Tolypocladium, Trichoderma, or Trichothecium.

In one embodiment the Filamentous fungus is F. sporotrichioides, such as NRRL 3299. In this pathway the production of farnesyl pyrophosphate (FPP) is conserved in these fungi and the Tri5 gene product, trichodiene synthase, is a terpene synthase enzyme responsible in this pathway for converting FPP to Trichodiene a C₁₅ (15 carbon atoms) isoprenoid. The Tri6 gene product is a positive transcription factor controlling the expression of the Tri5 gene product and FPP synthase in the isoprenoid pathway. The Tri10 gene produces a product which is a positive regulator for Tri5, Tri6, and FPP synthase in the Isoprenoid pathway. Both Tri6 and Tri10 appear to control the expression of FPP Synthase, HMG CoA reductase synthase, and Mevalonate kinase of the isoprenoid pathway and are responsible for up-regulating the flow of intermediates into the trichothecene pathway. In addition, both Tri6 and Tri10 are known to be active in the regulation of Tri5. The full or partial interruption of Tri5 gene has previously been shown in increasing the production of the isoprenoid products lycopene and beta-carotene on the order of 0.5 to 3 milligrams per gram of dry weight fungus under nitrogen limited batch culture conditions. Introducing multiple copies of these genes in a native strain background gives high levels of production for “trichothecenes”, while the interruption or enhancement of the Tri6 and Tri10 genes prior to the present invention have not been shown, let alone shown in combination with a Tri5 mutant. Prior to the present invention there has been no indication that combinations of these modifications would work together, let alone produce an improved or synergistic effect on the production of isoprenoids.

Tri5 gene encodes for the production of an enzyme for the conversion of FPP to trichodiene in the trichothecene biosynthetic pathway. The enzyme trichodiene synthase becomes the rate limiting step in the conversion of FPP. The Tri5 gene is also regulated by Tri6 and Tri10. The isolation and characterization of Tri5, Tri6, and Tri10 has shown that they all reside on an 8 kb DNA fragment in a gene cluster in F. sporotrichioides. It is known that they are located in similar positions in other trichothecene producing filamentous fungi.

Terpene synthase genes encode for enzymes for the conversion of isoprenoid pathway intermediates, such as geranyl pyrophosphate GPP (C₁₀), farnesyl pyrophosphate (FPP, C₁₅), and geranylgeranyl pyrophosphate (GGPP, C₂₀) to either linear or cyclic isoprenoid products. These genes exist in a number of organisms including bacteria, actinomycetes, marine invertebrates, filamentous fungi, plants, and algae where they are responsible for the production of hundreds of different isoprenoid products including, but not limited to, hydrocarbon products. Any suitable modified terpene synthase can be used in the present invention. For example, in one embodiment, the modified terpene synthase gene is aristolochene synthase (Ari1) from Penicillin roqueforti, as described in the Examples below.

The present invention relates to the production of C₁₀, C₁₅, and C₂₀ isoprenoid products (that can be used for pharmaceutical, cosmetic, perfume, pigment and colorant, fungicide, antiseptic, nutraceutical, biofuel, and fine chemical intermediate production) in a filamentous fungus having the isoprenoid pathway in sufficient quantities to be of commercial significance. By combining the disruption (biological or chemical) or partial blockage of Tri5 with the introduction of a modified terpene synthase gene and at least one other modification in Tri6 or Tri10 which leads to increased isoprenoid production commercial quantities of isoprenoids can be produced and isolated from the isoprenoid pathway in a filamentous fungus. The modification can be the addition or deletion of all or a portion of the genes, the substitution of other genes, for example, genes found to have constitutive activity or any other modification known in the art, to increase the production or activity or other property of the gene as necessary. The production of isoprenoid fuel products in this species would then represent a tremendous improvement over production bacteria or other species since it can occur under aerobic conditions and hydrocarbon isoprenoid products undergo a phase separation with water making the process more cost efficient to deploy on small scale production facilities, such as an on-farm fuel production facilities or other location where the sugar or lignocellulosic material (a biomass) resides. In addition, since most of these fungal species are able to utilize a number of different cellulose, hemicelluloses, and sugar sources for production, they represent a practical improvement which allows use of lignocellulosic and other polysaccharide or protein feedstocks without the substantial addition of processing enzymes for the conversion to component sugars or lignocellulosic stock which usually make other processes too costly and labor intensive. A filamentous fungal production system greatly reduces the need for enzymes, if not eliminates it, thus providing a novel practical solution to biological production of fuels because it could be produced on a small scale locally and it could easily provide an effective solution to the problem of feedstock transportation costs and logistics which can be a bigger barrier in some cases than the production of the fuel itself for any method.

The present invention filamentous fungus has the Tri5 gene modified to reduce or eliminate the production of the Tri5 gene product trichodiene synthase. Without this enzyme FPP is not converted in the next step of the conversion process. It is clear that a chemical modification that blocks the utility of the enzyme or its production would serve the same purpose and is considered part of the means for blocking the production or activity of the enzyme.

The Tri5 modification/treatment is then combined with at least one modification to the Tri6 or Tri10 gene/gene product and a terpene synthase gene/gene product such that larger quantities of isoprenoid products can be produced. It has been determined that at least a quadruple mutant produces more isoprenoid product than any of the triple mutants and in some cases, synergistically so. The quadruple refers to Terpene synthase mutant, Tri5 mutant, Tri6 mutant, and Tri10 mutant. It is difficult to produce these mutants, and absent applicant's disclosure, it would not have been known that one could achieve such mutants or that they would work to improve isoprenoid production to a commercial level. Obviously multiple mutations in the genes could be combined as well to give even higher production of isoprenoids.

The modifications to the Tri5 gene are known. The modifications to the other gene sequences can be achieved by any of the known methods for gene modification to increase or decrease the activity of a gene product or the like. One skilled in the art armed with the knowledge of producing the dual mutants could easily, without undue experimentation, make such dual mutants.

Now referring to the drawings, FIG. 1 is a flow chart choosing the trichodiene production route in filamentous fungi having the isoprenoid production pathway. As can be seen, farnesyl pyrophosphate is reacted on by the Tri5 gene product to produce trichodiene. The Tri4 gene product then reacts with trichodiene to produce 2-hydroxytrichodiene which is further metabolized to trichothecenes. The Tri6 and Tri10 gene products act as regulatory controls in both the isoprenoid and trichodiene pathways, hence their combination with modifications to the production of the Tri5 gene product and the introduction of a foreign sesquiterpene synthase (C₁₅ terpene synthase) gene product leads to redirection of farnesyl pyrophosphate (FPP) into the production of commercial quantities of foreign isoprenoid products.

In FIG. 2 there is a general flow chart of the isoprenoid biosynthetic pathway. Many different isoprenoid products, including diesel and jet fuel type products, are produced in this pathway from GPP, FPP, and GGPP.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

The filamentous fungus Fusarium sporotrichioides NRRL 3299 is selected with a deleted sequence for Tri5 and thus, cannot produce the Tri5 gene product, and a modified foreign terpene synthase gene sequence. The accumulation of isoprenoids is observed. This organism is treated to modify the Tri10 gene to have constitutive activity, thus increasing the production of FPP, GPP, or GGPP and further increasing isoprenoid production.

### Example 2

The NRRL 3299 is again modified. This time both the Tri6 and Tri10 gene are modified such that the terpene synthase gene product is increased in production. In a related example the Tri6, Tri10, or both genes are made constitutively active.

### Example 3

The NRRL 3299 is again modified. This time both the Tri6 and Tri10 gene are modified and one or more additional copies of the foreign terpene synthase gene are introduced such that the terpene synthase gene product is increased in production. In a related example the Tri6, Tri10, or both genes are made constitutively active.

### Example 4

The NRRL 3299 is again modified. This time both the Tri 6 and Tri10 gene are modified and one or more additional copies of the foreign terpene synthase gene are introduced such that the terpene synthase gene product is increased in production. In a related example the Tri6, Tri10, or both genes are made constitutively active and the foreign terpene synthase gene is made inducibly active for Tri6, Tri10, or both genes.

### Example 5

The NRRL 3299 is again modified, this time using Tri6 and/or Tri10 genes from a different fungal species. Both the Tri6 and Tri10 genes are modified such that the terpene synthase gene product is increased in production. In a related example the Tri6, Tri10, or both genes are made constitutively active.

### Example 6

### Generating Expression Plasmids Encoding Tri6-PK, Tri10-P1, and Ari1-T5

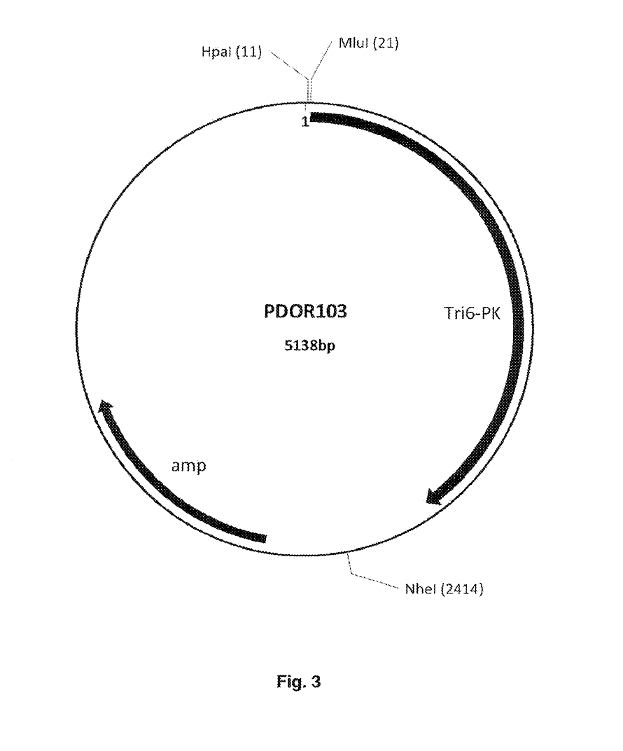

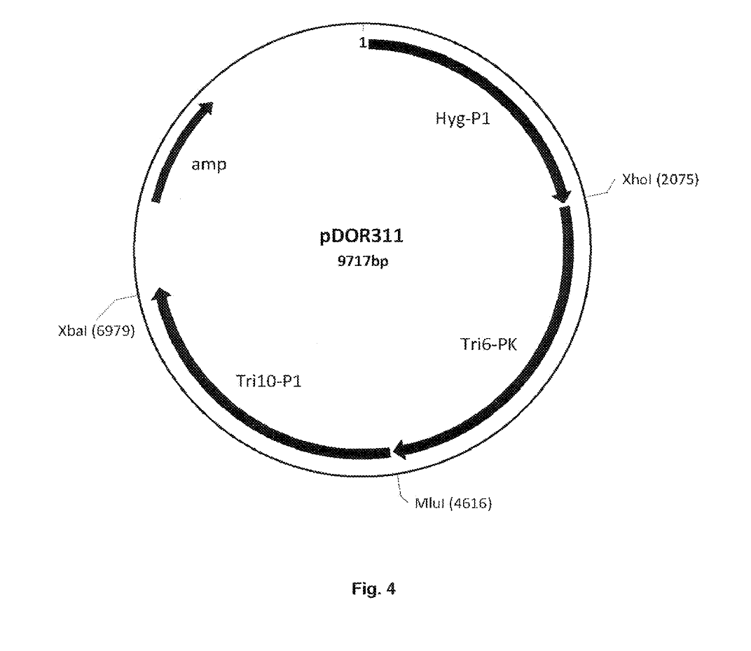

Expression plasmid pDOR311 was generated by inserting the Tri6-PK-Tri10-P1 gene fragment into the pDOR101 vector. Vector pDOR101 was generated by inserting a DNA synthesis construct comprising the Hyg-P1 gene into the EcoRV restriction site of pUC57 (GenBank accession number Y14837). Hyg-P1 consists of three genetic elements (Table 1) including hygromycin resistance selectable marker gene encoding the E. coli hygromycin phosphotransferase (GenBank accession number V01499) with the Cochliobolus heterostrophus P1 promoter sequence (GenBank accession number CCLPROA REGION: 1.645) and the Fusarium graminearum (teleomorph: Gibberella zeae) FgTri5 terminator sequence (GenBank accession number AF359361 REGION: 32132.32484). The Tri6-PK gene (SEQ ID NO: 1) was generated by DNA synthesis and cloned as a blunt ended fragment into the EcoRV restriction site of pUC57 to generate pDOR102. Tri6-P1 consists of the F. graminearum, FgTri6 coding region (GenBank accession number AF359361 REGION: 27401.28057), the F. graminearum FgTri5 terminator sequence, and the F. graminearum pyruvate kinase promoter sequence (GenBank accession number: FG10743.1 REGION: 3790933.3792134). The Tri10-P1 gene (SEQ ID NO: 2) was generated by DNA synthesis and cloned as a blunt ended fragment into the EcoRV restriction site of pUC57 to yield pDOR103 (FIG. 3). Tri10-P1 consists of the F. graminearum, FgTri10 coding region (GenBank accession number AF359361 REGION: 32799.34151) in which two conservative C to T nucleotide changes were introduced at positions 570 and 771 of the coding sequence designed to eliminate two consensus Tri6 DNA binding sites (YNAGGCC) proposed to function in the negative regulation of Tri10 gene expression (Tag, A. G.; Garifullina, G. F.; Peplow, A. W.; Ake Jr., C.; Phillips, T. D.; Hohn, T. M.; & Beremand, M. N. (2001) A Novel Regulatory Gene, Tri10, Controls Trichothecene Toxin Production and Gene Expression, Appl. Environ. Microbiology, 67: 5294-5302), the F. graminearum Tri5 terminator sequence, and the Cochliobolus heterostrophus P1 promoter sequence. To create the Tri6-PK-Tri10-P1 fragment pDOR102DNA was digested to completion with the restriction enzymes XbaI and MluI the reaction mixture resolved by gel electrophoresis, and the 1.7 kb Tri6-PK fragment was gel extracted. The isolated fragment was ligated with pDOR103DNA digested with restriction enzymes SpeI and MluI to generate plasmid pDOR203. The pDOR203DNA was digested to completion with the restriction enzymes XhoI and NheI, the reaction mixture resolved by gel electrophoresis, and the 4.9 kb Tri6-PK-Tri10-P1 fragment was gel extracted. The isolated fragment was ligated into XhoI XbaI digested pDOR101 yielding expression plasmid pDOR311. The nucleotide sequence of pDOR311 is given in SEQ ID NO: 3 and a plasmid map in FIG. 4.

TABLE 1
Expression Plasmid Genetic Elements

| | | | GenBank |
|---|---|---|---|
| Genetic Element | Source | Function | Accession |
| Promoter 1 | Cochliobolus | Constitutive | M17304 REGION: |
| | heterostrophus | promoter | 1-645 |
| Hyg | Escherichia coli | Hygromycin B | V01499 REGION: |
| CDS | | phosphotransferase | 231 . . . 1256 |
| | | coding sequence | |
| | | (Selectable marker) | |
| PrAri1 | Penicillin | Ari1 mRNA coding | L05193 REGION: |
| CDS | roqueforti | sequence | 220 1348 |
| AnAMDS | Aspergillus | Acetamidase gene | M16371 |
| | nidulans | (Selectable marker) | |
| FgTri5 | F. graminearum | Tri5 transcription | AF359361 |
| term | | termination | REGION: |
| | | | 32132 . . . 32491 |
| FgTri10 | F. graminearum | Tri10 coding | AF359361 |
| CDS | | sequence | REGION: |
| | | | 32799 . . . 34151 |
| FsTri5 | F. sporotrichioides | Trichodiene | AF359360 |
| Prom | | synthase promoter | REGION: 27090 . . . 28079 |
| FsTri5TR | F. sporotrichioides | Truncated | AF359360 |
| | | trichodiene | REGION: 28000 . . . 28000 |
| | | synthase CDS | |
| FgTri6 | F. graminearum | Tri6 coding | AF359361 |
| CDS | | sequence | REGION: |
| | | | 27401 . . . 28057 |
| FgPK | F. graminearum | Pyruvate kinase | FG10743.1 |
| prom | | promoter | REGION: |
| | | | 3790934 . . . 3792134 |

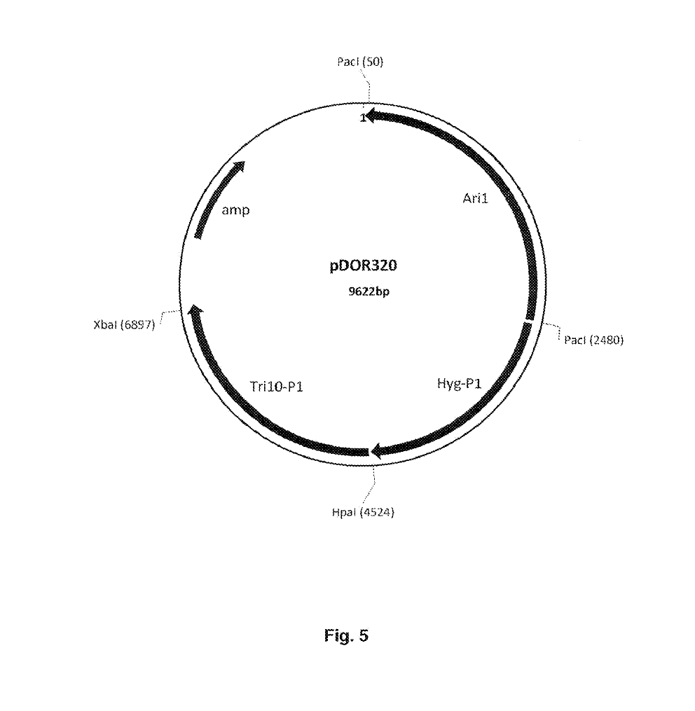

Expression plasmid pDOR320 was generated by first removing the Tri6-PK gene and then inserting the Ari1-T5 gene into pDOR311. The pDOR311 plasmid DNA was digested to completion with HpaI restriction enzyme the reaction mixture was resolved by gel electrophoresis, and the 7.2 kb fragment was gel extracted. The isolated fragment was self-ligated yielding expression plasmid pDOR313. The pDOR104 plasmid DNA was digested to completion with PspOMI and NotI restriction enzymes, the 2.45 kb fragment was gel extracted, and the isolated DNA fragment was ligated into the PspOMI restriction enzyme site of expression plasmid pDOR313 yielding expression plasmid pDOR320 with the Ari1-T5 gene in the opposite orientation as Hyg-P1. The nucleotide sequence of pDOR320 is given in SEQ ID NO: 4 and a plasmid map in FIG. 5.

### Example 7

### Generating a Disruption Plasmid Encoding FsTri5TR

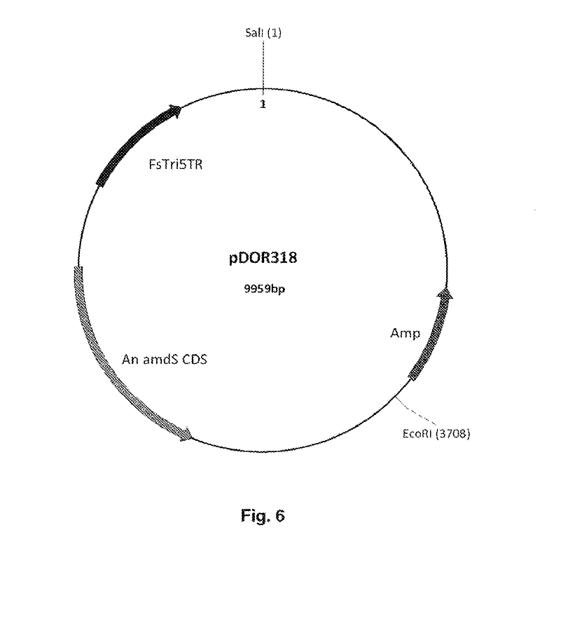

Disruption plasmid pDOR210 was generated by inserting FsTri5TR into the p3SR2 vector (Hynes et al. 1983. Mol. Cell. Biol. 3:1430-1439). Vector p3SR2 contains a SalI-EcoRI fragment (5,248 bp) of Aspergillus nidulans genomic DNA within which lies a 3,430 bp region identified as an acetamidase gene (AnAMDS, Table 1). FsTri5TR, a doubly truncated fragment of the FsTri5 CDS (Table 1) was generated by PCR amplifying from an F. sporotrichioides T-0927 genomic DNA template using primers DOR161 (SEQ ID NO: 5) and DOR163 (SEQ ID NO: 6). The 5′ truncated end of FsTri5TR starts at 61 bp downstream from the FsTri5 ATG and extends to 1065 bp downstream from the FsTri5 ATG. The PCR product was digested to completion using PstI restriction enzyme, the reaction mixture was resolved by gel electrophoresis, the 1.0 kb DNA fragment was gel extracted, and the isolated DNA fragment was ligated into the NsiI restriction enzyme site of p3SR2 to generate disruption plasmid pDOR210. Transformation of F. sporotrichioides T-0927 with pDOR210 leads to disruption of FsTri5 and loss of Tri5 function (Fusarium sporotrichioides T-0927 Tri5⁻) when FsTri5TR integrates into the genome via homologous integration. The nucleotide sequence of pDOR318 is given in SEQ ID NO: 7 and a plasmid map in FIG. 6 and is made similarly to pDOR210.

### Example 8

This example describes the generation of Fusarium sporotrichioides host strains useful in the invention.

The host strains were created by transforming Fusarium sporotrichioides T-0927 (NRRL 18340, obtained from Pennsylvania State University, Fusarium Research Center) parent cells with one of the expression plasmids of Example 6. DNA-mediated transformations into F. sporotrichioides T-0927 protoplasts were conducted using the polyethylene glycol procedure as described by (Royer, J. C.; Moyer, D. L.; Reiwitch, S. G.; Madden, M. S.; Jensen, E. B.; Brown, S. H.; Yonker, C. C.; Johnstone, J. A.; Golightly, E. J.; Yoder, W. T.; and Shuster, J. R. 1995). Fusarium graminearum A 3/5 as a novel host for heterologous protein production. Nature Biotechnology 13:1479-1483). For hygromycin selection, transformed host cells were initially grown in petri plates of agar medium (0.1% casein enzyme hydrolysate, 0.1 percent yeast extract, 1.6 percent agar, and 1 M sucrose) and after 24 hours a 1 percent water agar overlay containing 50 μg/mL of the antibiotic hygromycin was added to select transformants that integrated the expression plasmid DNA. Single colonies growing through the overlay after 3 to 10 days were transferred to V8 juice agar (per liter: 180 mL V8 juice, 800 mL water, 2 g CaCO₃, and 15 g Bacto agar) containing hygromycin (150 μg/mL) and cultures were grown at 28 degree C. for 7 to 10 days and then conidia were harvested in sterile water. The conidia were stored at −80.degree. C. in cryo-vials in 1 mL stock aliquots made up of 200 μL sterile 50% glycerol and 800 μL suspension of conidia. For acetamidase selection, transformed host cells were grown in petri plates of agar medium containing 10 mM acetamide, and 15 mM cesium chloride, Cove salts (Cove, D. J., Biochem. Biophys. Acta 113:51-56, 1966) and 0.8 M sucrose. After 10 to 14 days single colonies were transferred to V8 juice agar (per liter: 180 mL V8 juice, 800 mL water, 2 g CaCO₃, and 15 g Bacto agar) and cultures were grown at 28 degree C. for 7 to 10 days and then conidia were harvested in sterile water. The conidia were stored at −80.degree. C. in cryo-vials in 1 mL stock aliquots made up of 200 μL sterile 50% glycerol and 800 μL suspension of conidia. All gene integrations in transformants were confirmed by phenotypic analysis and polymerase chain reaction (“PCR”) analysis of genomic DNA for DNA fragments representing the integrated genetic elements.

### Example 9

This example demonstrates production of aristolochene in host strains expressing Tri10-P1 as compared to production by the parent strain Fusarium sporotrichioides T-0927 Tri5⁻.

Inoculum cultures of each host strain was established by growing a stock aliquot of each strain on V8 agar medium with hygromycin (150 μg/mL) for 7 to 10 days. Conidia were harvested from inoculum cultures using cell scrapers and used to inoculate at an initial number of 1×10⁵ spores/mL in separate 125 mL flasks containing 62.5 mL of GYEP medium (0.1 percent Bacto yeast extract, 0.1 percent Bacto peptone, and 5 percent glucose). Cultures were incubated at 28 degree. C. on a rotary shaker at 200 RPM for 24 hours at which point they were overlain with 6.25 mL of dodecane. After 168 hours 45 mL of culture material enriched for the organic layer was transferred to a 50 mL centrifuge tube and centrifuged for 5 min at 5000×g after which samples of the organic overlay layer were taken.

A volume of 4 μl of the organic overlay sample was added to 996 μl of isopropyl alcohol containing caryophyllene as an internal standard in a clean glass vial prior to analysis. Samples were analyzed on a Hewlett-Packard 6890 gas chromatograph (GC) coupled to a 5973 mass selective detector (MSD) outfitted with a 7683 series injector and autosampler and equipped with an Zebron ZB-Wax plus wax capillary column (0.25 mm i.d.×30 m with 0.25 mm film) (available from Agilent Technologies). For all experiments, needle sampling depth was set to 8 mm. The GC was operated at a He flow rate of 2 mL min¹, and the MSD operated at 70 eV. Splitless injections (2 μL) were performed with an injector temperature of 250° C. The GC was programmed with an initial oven temperature of 50° C. (5-min hold), which is then increased 10° C. min¹ up to 180° C. (4-min hold), followed by a 100° C. min¹ ramp until 240° C. (1-min hold). A solvent delay of 8.5 min was included prior to the acquisition of MS data. Product peaks are quantified by integration of peak areas using Enhanced Chemstation (version B.01.00, Agilent Technologies). Aristolochene was identified based on its published aristolochene mass fragmentation profile (Felicetti, B. and Cane, D., J. Am. Chem. Soc. 126 7212-7221, 2004) and had a retention time of 17.2 minutes using this GC protocol. Caryophyllene was used as a standard for quantitation and had a retention time of 15.92 minutes. A response factor was established for caryophyllene based on the GC peak area/mg/mL where a caryophyllene peak area corresponding to a concentration of 1.0 mg/mL equals 1.0 CP unit. Aristolochene titer was calculated as the ratio of the peak area for aristolochene to the peak area of the caryophyllene response factor and reported in CP units.

After 120 hours of growth, host strains N18 and N23 were found to produce 2.8 CP units aristolochene/mL culture medium and 3.1 CP units aristolochene/mL culture medium, respectively. Both strains producted 0.0 CP units trichodiene/mL culture medium Parent strain Fusarium sporotrichioides T-0927 Tri5⁻cultures was found to produce 0.0 CP units aristolochene/mL culture and 0.0 CP units trichodiene/mL culture.

TABLE 2
Host strain Plasmid Source

| | | | Fungal |
|---|---|---|---|
| Host | | Expression | Antibiotic |
| strain | Parent Strain | Plasmids | Selection |
| N18 | F. sporotrichioides | pDOR320 | Hygromycin |
| | T-0927 (Tri5⁻) | | |
| N23 | F. sporotrichioides | pDOR320 | Hygromycin |
| | T-0927 (Tri5⁻) | | |

1. A mutant isoprenoid producing filamentous fungus having the trichothecenes pathway comprising:
a) a disrupted Tri5 gene, or a mutant Tri5 gene having low trichodiene synthase production; and
b) a modified Tri6 gene, a modified Tri10 gene, or a modified gene encoding a terpene synthase, the gene modified to increase production of the gene product;
wherein the mutant filamentous fungus produces at least 10% more isoprenoid product than the parent filamentous fungal cell when cultured under the same conditions.

2. The mutant filamentous fungus according to claim 1 wherein the modified gene has been modified to have inducible activity in producing a gene product.

3. The mutant filamentous fungus according to claim 1 wherein the modified gene has been modified to have constitutive activity in producing a gene product.

4. The mutant filamentous fungus according to claim 1 wherein the filamentous fungus is selected from the group consisting of Acremonium, Aspergillus, Aureobasidium, Cryptococcus, Filibasidium, Fusariuni, Gibberella, Humicola, Magnaporthe, Mucor, Myceliophthora, Myrothecium, Neocallimastix, Neurospora, Paecilomyces, Penicillium, Piromyces, Schizophyllum, Stachybotrys, Talaromyces, Thermoascus, Thielavia, Tolypocladiurn, or Trichoderma strain.

5. The mutant filamentous fungus according to claim 4 wherein the filamentous fungus is a Fusarium species.

6. The mutant filamentous fungus according to claim 5 wherein the Fusarium species is Fusarium sporotrichioides.

7. The mutant filamentous fungus according to claim 5 wherein the Fusarium species is Fusarium venenatum.

8. The mutant filamentous fungus according to claim 1 comprising at least two modified genes selected from a modified Tri6 gene, a modified Tri10 gene, and a modified gene that encodes a terpene synthase, the genes modified to increase the production of the gene products.

9. The mutant filamentous fungus according to claim 1 wherein there is a nonrevertable site-selected deletion of part or all of nucleic acid coding for the Tri5 gene product such that the Tri5 gene is inactivated.

10. The mutant filamentous fungus according to claim 1 wherein there is a nonrevertable site-selected deletion or modification to the nucleic acid sequence encoding for the Tri5 gene product such that the Tri5 gene product enzymatic or catalytic activity is reduced by at least 10 percent when compared to the parent strain under the same conditions.

11. The mutant filamentous fungus according to claim 1 wherein the modified gene of b) comprises more than one copy of the nucleic acid sequence encoding for the gene product.

12. The mutant filamentous fungus according to claim 11 wherein at least one of the additional copies of the nucleic acid sequence encoding for the gene product is in a vector which is capable of autonomous maintenance in the filamentous fungus.

13. The mutant filamentous fungus according to claim 1 wherein the modified gene of b) comprises a coding sequence operably linked to a promoter from a Tri6 or Tri10 inducibly active filamentous fungal gene.

14. The mutant filamentous fungus according to claim 1 wherein the modified gene of b) comprises a coding sequence operably linked to a promoter from a constitutively active filamentous fungal gene.

15. The mutant filamentous fungus according to claim 1 wherein the modified gene of b) comprises a coding sequence operably linked to a foreign promoter.

16. (canceled)

17. A mutant isoprenoid producing filamentous fungus having the trichothecenes pathway comprising:
a) a modified Tri6 gene, a modified Tri10 gene, or a modified gene encoding a terpene synthase, the gene modified to increase production of the gene product; and
b) the presence of a Tri5 inhibitor sufficient to inhibit at least a portion of the Tri5 gene product;
wherein the mutant filamentous fungus produces at least 10 percent more isoprenoid than the parent filamentous fungal cell when cultured under the same conditions.

18-22. (canceled)

23. A method of producing an isoprenoid comprising:
a) cultivating the mutant filamentous fungus of claim 1 and;
b) isolating the isoprenoid.

24. (canceled)

25. The method according to claim 23 wherein the mutant filamentous fungi produces at least 0.25 g of isoprenoid per gram of glucose consumed.

26-29. (canceled)

30. The method according to claim 23 wherein the mutant filamentous fungus is cultivated in media prepared from biomass.

31. The method according to claim 23 wherein the mutant filamentous fungus is cultivated in media prepared from lignocellulosic feedstock.

32. (canceled)

33. The mutant filamentous fungus according to claim 1 comprising a modified Tri6 gene, the gene modified to increase production of the gene product.

34. The mutant filamentous fungus according to claim 1 comprising a modified Tri10 gene, the gene modified to increase production of the gene product.

35. The mutant filamentous fungus according to claim 1 comprising a modified gene that encodes a terpene synthase, the gene modified to increase production of the gene product.

36. The mutant filamentous fungus according to claim 1 comprising a modified gene that encodes a terpene synthase, and further comprising a modified Tri6 gene or a modified Tri10 gene, the genes modified to increase production of the gene products.

37. The mutant filamentous fungus according to claim 1 comprising a modified Tri10 gene and a modified gene that encodes a terpene synthase, the genes modified to increase production of the gene products.

38. The mutant filamentous fungus according to claim 1 wherein the mutant is capable of producing at least 0.25 g of isoprenoid per gram of glucose consumed.
